(19) United States
(12) Patent Application Publication
(10) Pub. No.: US 2026/0224792 A1
(43) Pub. Date: Aug. 6, 2026

(54) VASCULAR ACCESS SYSTEM AND METHOD OF USE

(71) Applicant: Merit Medical Systems, Inc., South Jordan, UT (US)

(72) Inventor: Jackson Taggart, Salt Lake City, UT (US)

(21) Appl. No.: 19/463,782

(22) Filed: Jan. 29, 2026

Related U.S. Application Data
(60) Provisional application No. 63/752,324, filed on Jan. 31, 2025.

(51) Int. Cl. A61M 1/36; A61M 25/00; A61M 39/10

(52) U.S. Cl. CPC A61M 1/3655; A61M 25/005; A61M 39/10; A61M 2025/0004

(57) ABSTRACT

A vascular access system is provided for providing blood flow between two locations in a vascular system of a patient. The vascular access system includes a fluid conduit configured to be fluidly coupled to a first location and a second location in the vascular system of the patient. The fluid conduit includes a peripheral portion comprising a first inner diameter, and a central portion comprising a second inner diameter that is smaller than the first inner diameter.

### RELATED APPLICATIONS

This application claims priority to United States Provisional Application No. 63/752,324, filed on January 31, 2025 and titled, “Vascular Access and Method of Use,” which is hereby incorporated by reference in its entirety.

### TECHNICAL FIELD

The present disclosure relates to systems for achieving vascular access and methods of using the same. More particularly, some embodiments relate to a vascular access system for performing hemodialysis.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 illustrates a vascular access system (VAS) for forming an arteriovenous (AV) anastomosis, according to embodiments described herein.

FIG. 2 illustrates an exploded view of a connection site of the VAS of FIG. 1, according to embodiments described herein.

FIG. 3 illustrates a cutaway view of components of the VAS of FIG. 1, according to embodiments described herein.

FIG. 4 illustrates a cutaway view of a venous outflow component (VOC) of the VAS of FIG. 1, according to embodiments described herein.

FIG. 5 illustrates a cutaway view of a graft component and a conduit connector of the VAS of FIG. 1, according to embodiments described herein.

### DETAILED DESCRIPTION

In the United States, approximately 400,000 people have end-stage renal disease requiring chronic hemodialysis. Permanent vascular access sites for performing hemodialysis may be formed via a vascular access system (VAS) including an arteriovenous (AV) anastomosis whereby a vein is attached to an artery to form a high-flow shunt or fistula. A vein may be directly attached to an artery, but it may take 6 to 8 weeks before the venous section of the fistula has sufficiently matured to provide adequate blood flow for use with hemodialysis. Moreover, a direct anastomosis may not be feasible in all patients due to anatomical considerations. Other patients may require the use of artificial graft material to provide an access site between the arterial and venous vascular systems.

Although many materials that have been used to create prosthetic grafts for arterial replacement have also been tried for dialysis access, expanded polytetrafluoroethylene (ePTFE) is often used. The reasons for this include its ease of needle puncture and particularly low complication rates (pseudo-aneurysm, infection, and thrombosis). However, AV grafts still require time for the graft material to mature prior to use, so that a temporary access device, such as a catheter, must be inserted into a patient for hemodialysis access until the AV graft has matured.

The use of temporary catheter access exposes the patient to additional risk of bleeding and infection, as well as discomfort. Also, patency rates of ePTFE access grafts may not be satisfactory, as the overall graft failure rate remains high and failure rates are further increased in higher-risk patients, such as diabetics. These access failures result in disruption in the routine dialysis schedule and create hospital costs of over $2 billion per year.

Research indicates that graft failures from localized stenosis at the venous end of AV grafts are may be due to intimal hyperplasia, compliance mismatch between the graft and the native vein anastomosis, and turbulent flow at the anastomosis site.

Although these devices may be constructed as a single-piece, integrated device, a multi-piece device comprising separate components that are later joined together may also be designed. A multi-component device may have several advantages for certain therapies. First, a multi-piece device allows switch-out of one or more components of the device. This allows the tailoring of various device characteristics to the particular anatomy and/or disease state, for instance, by using components of different dimensions.

This also reduces the cost of treating patients in several ways. It reduces the amount of inventory of a given device by stocking an inventory range of components, rather than an inventory range of complete devices. Also, if an incorrect device is initially selected for use in a patient, only the incorrect component is discarded, rather than the entire device. Further, separate multiple components of a device may be easier to manufacture compared to an integrated form of the device. Additionally, it may be easier for a physician to implant separate components of a device and then join them together rather than implanting an integrated device. It may also allow the components to be trimmable as needed to accommodate various patient anatomies. An integrated device may be excessively bulky and can slow the implantation procedure, thereby increasing operating room time and costs as well as increasing the risk of physician error.

One challenge with multi-component devices includes managing potential sources of turbulent flow along the flow path of blood through the device. For example, sharp indentations or protrusions within the lumen of the device cause alterations in flow at the interface that may result in hemolysis and clot formation. Such interfaces create an increased risk of creep or separation of joined components over time that can worsen the flow characteristics at the interfaces or even result in loss of flow, respectively.

This can be a particular concern when transitioning between components with different internal diameters, such as when a graft and catheter have varying lumen sizes. For example, maintaining a smaller inner diameter in the venous outflow component (VOC) relative to the arterial graft (AG) is often beneficial. However, abrupt changes in lumen diameter, i.e., from the AG to the VOC, can cause turbulent flow and lead to thrombus formation and stenosis.

Thus, various components may benefit from one or more design features that maintain smooth flow between components through the interface and also resist creep or separation of the joined components. Such a connector system may be used with AV grafts, peripherally inserted central catheters (PICC), implantable infusion catheters with and without fluid reservoirs, implantable infusion pumps, left ventricular assist devices, and any other device where providing laminar flow between two body fluid conduits may be beneficial. The connectors may also be integrated with such conduit or reservoir containing devices.

Aspects and implementations of the present disclosure address the above and other challenges by providing a VOC component with a tapering or gradually diminishing inner lumen. Such a VOC component can provide an elongated or gradual reduction in diameter of the inner lumen, enhancing the consistency of the lumen profile and of laminar blood flow through the device.

The phrase “coupled to” is broad enough to refer to any suitable coupling or other form of interaction between two or more entities, including mechanical and fluidic interaction. Thus, two components may be coupled to each other even though they are not in direct contact with each other. The phrase “fluid communication” is used in its ordinary sense and is broad enough to refer to arrangements in which a fluid (e.g., a gas or a liquid) can flow from one element to another element when the elements are in fluid communication with each other.

The terms “proximal” and “distal” are opposite directional terms. As used herein, the distal end of a device or component is the end of the component that is furthest from the physician during ordinary use. The proximal end refers to the opposite end, or the end nearest the physician during ordinary use. For example, the proximal end of an introducer sheath used in minimally invasive vascular treatment is the end accessible to a practitioner during use, while the distal end is disposed within a patient’s vascular system when the sheath is placed into such a patient.

The terms “central” and “peripheral” are opposite directional terms. As used herein, the central end of a device or component refers to the end or portion closest to core structures of an anatomy, such as the heart or central circulation system. The peripheral end refers to the end directed or furthest away from these core structures. E.g., toward outer regions of the anatomy, such as the limbs. For example, the central end of a vascular access system may be positioned near the heart, while the peripheral end is positioned close or closer to a limb when the device is deployed in a patient.

Embodiments may be understood by reference to the drawings, wherein like parts are designated by like numerals throughout. The components of the embodiments as generally described and illustrated in the figures herein can be arranged and designed in a wide variety of different configurations. Thus, the following more detailed description of various embodiments, as represented in the figures, is not intended to limit the scope of the present disclosure but is merely representative of various embodiments. While various aspects of the embodiments are presented in drawings, the drawings are not necessarily drawn to scale unless specifically indicated.

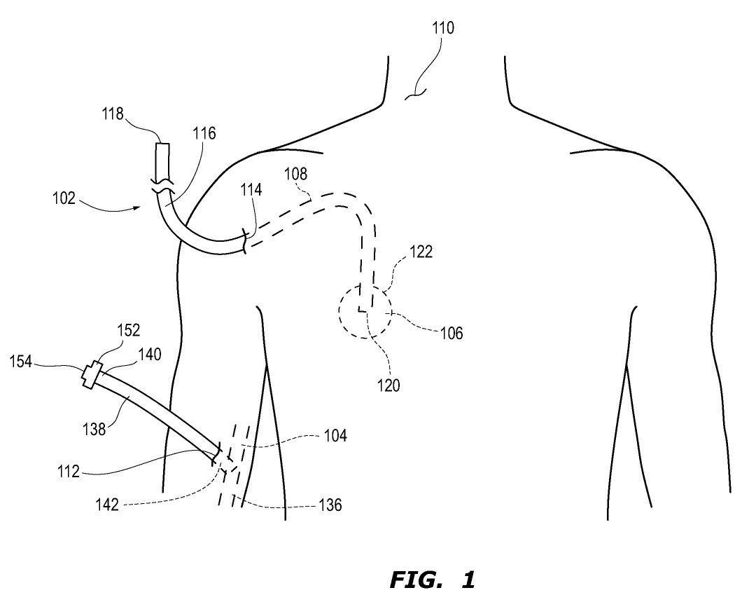

FIG. 1 illustrates a vascular access system (VAS) 102 for forming an arteriovenous (AV) anastomosis, according to embodiments described herein. As seen in the illustrated embodiment, VAS 102 includes a vascular outflow component (VOC) 116, a graft component 138, and a conduit connector 152.

VAS 102 can form a continuous lumen between an anastomosis site (e.g., at artery 136) and a venous outflow location 122. During placement, VOC 116, and graft component 138 can be placed separately and be in an open configuration as seen in FIG. 1. Graft component 138 and VOC 116 can be connected via conduit connector 152 and be in a closed, or fluidly connected configuration.

In some embodiments, graft component 138 can feature two ends: a peripheral end 142 and a central end 140. Peripheral end 142 can be attached to a first vascular segment or location (e.g., artery 136, e.g., the brachial artery of a patient). Graft component 138 can be made of biocompatible materials suitable for long-term implantation, such as expanded polytetrafluoroethylene (ePTFE), which provides flexibility and durability.

Conduit connector 152 can fluidly connect graft component 138 and VOC 116 to form a lumen 108 of VAS 102. In certain embodiments, conduit connector 152 can be positioned between graft component 138 and VOC 116 such that central end 140 of graft component 138 is connected to a peripheral portion of conduit connector 152, and a peripheral end 118 of VOC 116 is connected to a central portion 154 of conduit connector 152.

In some embodiments, once formed, continuous lumen 108 of VAS 102 can provide a conduit or pathway configured to shunt blood from a first vascular segment or location to a second vascular segment or location. For instance, in some cases, VAS 102 can be used to fluidly connect an artery 136 to a venous outflow location 122 (e.g., the internal jugular (IJ) vein of the patient).

Thus, VAS 102 includes a central end 106 which can be formed by a central end 120 of VOC 116 and can be configured to be inserted into a venous vascular segment (e.g., the IJ vein of a patient). Peripheral end 142 can be formed by a peripheral portion of a graft component 138 and can be adapted or configured to couple with an arterial vascular segment (e.g., the brachial artery of a patient). Once set in place, lumen 108 of VAS 102 can be accessed from outside the patient (e.g., via a needle puncturing graft component 138) to facilitate dialysis or other treatments.

VOC 116 can be designed to be inserted into a second vascular segment, such as the IJ vein, and advanced into the superior vena cava or right atrium. Peripheral end 118 of the VOC 116 can be trimmed to the desired length during the implantation procedure. Before connecting to the conduit connector 152, any reinforcing materials at the trimmed end, such as nylon windings, are removed to allow for a snug fit.

In one embodiment, to place VAS 102 within a patient, the patient can first be prepped and draped in the usual sterile fashion. Either local or general anesthesia can be achieved. The brachial artery of the patient can then be palpated, and terminal access site 112 can be marked. The IJ vein can be located, and an initial access site 110 to the IJ vein can be selected using anatomical landmarks and/or radiographic visualization such as ultrasound. In some case, a guidewire can be passed into the IJ vein, and a dilator can be passed over the guidewire to facilitate insertion of an introducer into the IJ vein. A small scalpel incision may be needed at the guidewire insertion site if the skin and/or subcutaneous tissue create excessive resistance to the insertion of the dilator. After the dilator is removed, an introducer can be inserted over the guidewire and into the IJ vein. The introducer may be a standard or custom type of introducer.

VOC 116 of VAS 102 can then be inserted into the introducer, through the IJ vein, and into the superior vena cava or right atrium. The position of central end 106 of VAS 102 and/or VOC 116 can then be confirmed radiographically, and the patient checked for accidental collapse of the lung due to improper insertion. The introducer can then be removed, either by pulling the introducer over the proximal end of the catheter section, if possible, or by peeling away the introducer if a peel-away introducer was provided.

A surgical rod can then be inserted into the subcutaneous space through initial access site 110. The rod can be used to subcutaneously tunnel toward the anterior shoulder. In some embodiments, subcutaneous tunneling and implantation of VAS 102 may occur generally simultaneously. Once the anterior shoulder is reached, a scalpel can be used to create an intermediate access site 114 to the rod. The rod can then be removed from the initial access site 110, and peripheral end 118 of VOC 116 can be passed through the subcutaneous pathway to exit from intermediate access site 114.

The same surgical rod, or a different rod, can then be inserted into intermediate access site 114 and used to subcutaneously tunnel distally down the arm until the marked brachial artery site is reached. Terminal access site 112 to the rod can be created and further exposed to access the brachial artery.

Peripheral end 142, of graft component 138 can then be attached to the brachial artery. Alternatively, the anastomosis may be performed after graft component 138 is subcutaneously positioned.

Central end 140 of graft component 138 and conduit connector 152, can then be passed from terminal access site 112 to intermediate access site 114. In some cases, a connector sleeve with integrated strain relief structure can be passed over peripheral end 118 of VOC 116. Initial and terminal access sites 110, 112 can then be checked for any redundant conduit and pulled taut from the intermediate access site 114 if needed. As previously discussed, peripheral end 118 of VOC 116 can be trimmed to the desired length. In some cases, about 0.5 cm to about 1 cm segment of nylon winding at the trimmed end of the catheter section can be separated and cut away.

In some cases, peripheral end 118 of VOC 116 is fitted to the conduit connector 152 which is fitted to graft component 138. VOC 116 can then be secured to conduit connector 152. The exposed portions of conduit connector 152, attached to central end 140 of the graft component 138 and peripheral end 118 of VOC 116, are either pulled from the graft end or pushed into the subcutaneous space through intermediate access site 114. Blood flow through VAS 102 is confirmed either by palpation or preferably by ultrasound and/or angiography. The three access sites are sutured closed. The implanted VAS 102 is then accessed with hemodialysis needles to perform hemodialysis. For example, graft component 138 may be pierced by hemodialysis needles to perform hemodialysis. Graft component 138 may comprise a self-sealing element that would reseal after the needle punctures and is removed from graft component 138.

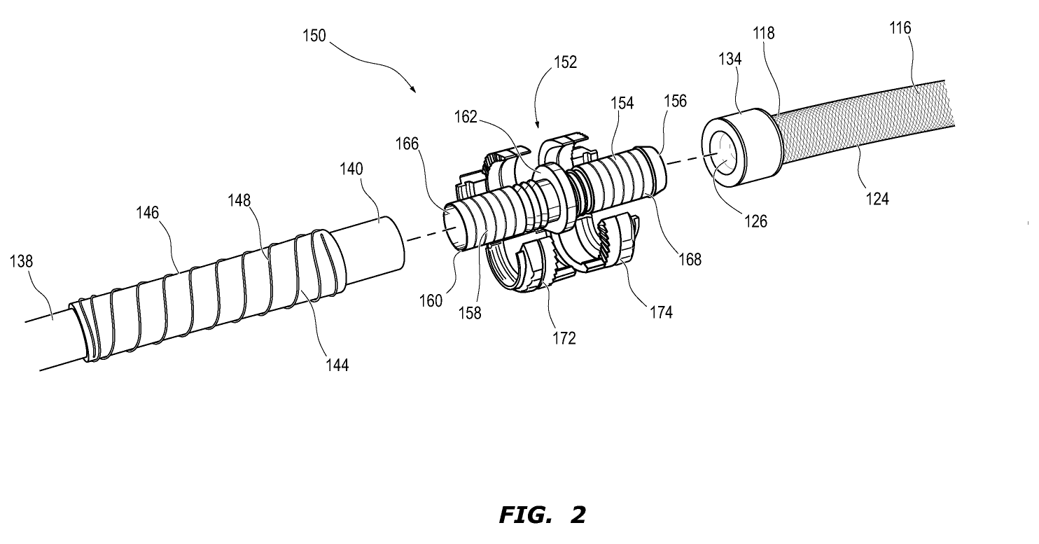

FIG. 2 illustrates an exploded view of an embodiment of a connection site 150, including conduit connector 152 of VAS 102 of FIG. 1, according to embodiments described herein.

As seen in the illustrated embodiment, connection site 150 includes conduit connector 152, graft component 138, and VOC 116.

In some cases, graft component 138 can take a variety of configurations, for example, having wall thickness in a relatively wide range, but otherwise be similar to a vascular graft. Graft component 138 can be connected to and in fluid communication with peripheral portion 158 of conduit connector 152. Central portion 154 of conduit connector 152 can be connected to peripheral end 118 of VOC 116.

The central end 140 of graft component 138 can be configured to mate with conduit connector 152. Before assembly, central end 140 may be radially expanded using a dilator to accommodate the diameter of the conduit connector 152. The inner surface of central end 140 may have engagement features or textures that correspond to complementary features on the conduit connector 152, enhancing the mechanical bond between the two components.

Additionally, conduit connector 152 can include a shoulder 162 near its midpoint. In some embodiments, shoulder 162 can act as a physical stop for graft component 138 (and/or VOC 116), ensuring that it is advanced to the correct position during assembly. An internal lumen 166 of conduit connector 152 can be smooth and continuous, maintaining an unobstructed pathway for blood flow and minimizing turbulence or areas of stasis that could lead to thrombosis.

In some embodiments, central portion 154 of conduit connector 152 can have a tubular structure with one or more engagement features 168 to enhance the security of connection or provide mechanical engagement between the conduit connector 152 and the VOC 116. Engagement features 168 can have enlarged outer diameters or perimeters greater than the diameter of the tubular structure of central portion 154 of the conduit connector 152.

In some embodiments, engagement features 168 can comprise one or more barbs. As illustrated, in some embodiments, engagement features 168 can have conical shapes with the outer perimeter or diameter increasing from a first end to a second end of engagement features 168. In some embodiments, connection site 150 can have a clamshell structure formed by first member 172 and second member 174. The term clamshell structure is a broad term intended to cover a combination of a plurality of members, at least one of which can pivot away from and toward the other, wherein when pivoted together a closed configuration is formed, and is not limited to structures with clamshell type shapes or edge-to-edge contact around a perimeter. For example, in some embodiments, the hinge-type mechanism can have arcuate finger-shaped members or have members with outer diameters or surfaces that are more angular or square in shape and inner diameters or surfaces more tubular or cylindrical in shape. In this manner, connection site 150 can include any mechanism or configuration that performs its intended function.

During assembly, a hub 134 of peripheral end 118 of VOC 116 can be fitted over central portion 154 of conduit connector 152. Similar to graft component 138, VOC 116 may require radial expansion using a dilator to accommodate the connector's diameter into lumen 126 of VOC 116. The connection can be secured using a crimp ring or clamping mechanism, which compresses VOC 116 against conduit connector 152, ensuring a leak-proof and durable bond. In some embodiments, VOC 116 may not have hub 134 and peripheral end 118 of VOC 116 may couple directly to central portion 154 of conduit connector 152.

In some embodiments, the system or assembly can be provided with one or more kink-resistant elements 144 surrounding or adjacent to central end 140 of graft component 138 (and/or VOC 116, not shown in FIG. 2). In some embodiments, kink-resistant element 144 can include both a resilient characteristic and a soft inner surface. For example, a springy material or configuration, such as a nitinol coil (e.g., coil 148) can be provided to resist unwanted, unpredictable deformation in the zone of kink-resistant element 144. Also, a soft material or construction, such as a silicone sleeve (e.g., sleeve 146) can be provided to isolate graft component 138 from pinching due to kinking.

Accordingly, in some cases, kink-resistant element 144 can be or include a coil 148. In some cases, coil 148 is preferably wound sufficiently tightly such that adjacent turns of the wires touch each other. This structure substantially prevents compression of the coil, which during implantation, substantially prevents tissue from being caught between adjacent turns of the coil or prevents the tissue from compressing the coil axially.

As discussed, in some embodiments, kink-resistant elements 144 can be or include an elastomeric sleeve 146 that can be slid over a portion of the first end of the connector as well as be or include coil 148 that surrounds the inflow component. Sleeve 146 can enhance kink-resistant element 144 in reducing or minimizing strain on the inflow component by the connector. Thus kink-resistant element 144 can reinforce the connection point, provide additional mechanical stability, and protect against kinking or bending that could compromise the lumen.

In some cases, sleeve 146 can be formed from a flexible material such as silicone and can be disposed within kink-resistant element 144, such that sleeve 146 can have an outer diameter substantially the same as the inner diameter of kink-resistant element 144. In some embodiments, sleeve 146 can extend beyond a terminal end of kink-resistant element 144.

In some cases, the kink-resistant element 144 can comprise a resilient structure, such as a nitinol coil defining a lumen therein of substantially constant diameter. Other materials that can be used include PEEK, stainless steel, MP35N and other similar metals.

In one embodiment sleeve can include an elastomeric configuration, such as a cylindrical ring of silicones or polyurethanes. In one embodiment, the silicone can have a hardness of approximately 50 durometer (Shore A). Further details of the elastomeric configuration are discussed above.

As illustrated in FIG. 2, conduit connector 152 is in an open configuration. In some embodiments, a connection mechanism, such as that provided by first member 172 and second member 174 can be integrated with conduit connector 152. In other embodiments, the connection mechanism can be a separate component from conduit connector 152. Conduit connector 152 can be configured with a flange or protrusion to which a connection mechanism such as a hinge structure and/or a pin can be mounted. The flange can be configured as a radially projected member of conduit connector 152, in some embodiments forming the widest portion of a profile of conduit connector 152.

In the illustrated embodiment, a first and second member 172, 174 form a connection mechanism configured to closable and to engage portions of an outer surface of graft component 138 (and/or VOC 116) to secure it to conduit connector 152. A range of positions or closed configurations for first and second member 172, 174 are provided such that a range of thicknesses of grafts can be secured to conduit connector 152. The ability to accept ranges of thicknesses enables conduit connector 152 to work with different distributions of products, e.g., having average wall thicknesses at least at their distal ends that are significantly different. As discussed elsewhere herein, this feature advantageously may enable a system including conduit connector 152 to be used in more settings and with various different products in a product line.

In some embodiments, first and/or second members 172, 174 of can be provided with protrusions, gripping structures or teeth on mating surfaces that maintain compressive force on graft component 138 and/or VOC 116 in the closed configurations. For instance, teeth (not shown in FIG. 2) can be formed along an arcuate body on the first and second member 172, 174 allowing a secured or locked engagement at any position over an arcuate path of movement of first and second member 172, 174. As discussed above, first and second member 172, 174 can be pivotably connected at one axis of rotation, such as by a pin.

Radiopaque materials may be included within the connector devices described herein. Examples of suitable material include but are not limited to: platinum, tantalum, tungsten, gold, palladium, iridium, barium sulfate, and any combination thereof. These marking materials may be doped into the molded materials or in the form of rings, patches, plates, wire, or other shapes. They may be distributed in the entire device or at one or both ends of the device or anywhere in between.

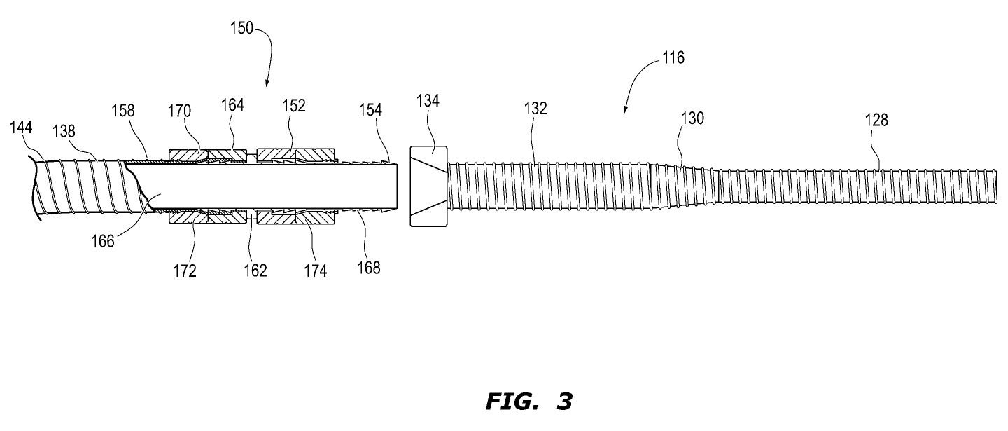

FIG. 3 illustrates a cutaway view of components of VAS 102 of FIG. 1, according to embodiments described herein. As seen in the illustrated embodiment, conduit connector 152 is in a closed configuration, and lumen 166 of conduit connector 152 extends from a peripheral portion 158 to a central portion 154 of conduit connector 152. In some embodiments, lumen 166 can have a constant diameter. A protrusion or shoulder 162 is circumferentially positioned around an outer wall of conduit connector 152.

Peripheral portion 158 of conduit connector 152 can extend from shoulder 162 to the peripheral end (not seen in FIG. 3) of conduit connector 152. Graft component 138 can be slid in an axial direction from peripheral portion 158 to central portion 154. Graft component 138 can be slid over the peripheral end until the central end of graft component 138 abuts with shoulder 162.

A nominal inner diameter of the above devices can be adapted and suitable for use with any graft or inflow component used for vascular access. Examples include 6 mm ID grafts as well as 5- and 7-mm ID grafts.

In some cases, VAS 102 can reduce or eliminate abrupt diameter transitions by maintaining a constant (e.g., 6 mm) internal diameter from graft component 138 to peripheral end 118 of VOC 116. A transition from the inner diameter of graft component 138 and/or lumen 166 to a smaller inner diameter of the VAS can occur gradually in VOC 116. Otherwise stated, a transition from a larger inner diameter (e.g., 6mm) to a smaller inner diameter (e.g., 5mm) can occur gradually along a tapering section 130 of VOC 116. Such a gradual taper can reduce turbulent flow, improve laminar blood flow through VOC 116, and improve long-term outcomes.

In some embodiments, tapering section 130 can be disposed between a peripheral section 132 and a central section 128 of VOC 116. For instance, in some cases, peripheral section 132 can be between 5-15 cm in longitudinal length. In some case, tapering section 130 can be between 4 and 8 cm in longitudinal length. In some cases, central section 128 can be between 10-20 cm in longitudinal length. In some cases, tapering section 130 is less than 20 percent of the length of VOC 116.

In some cases, the inner diameter or perimeter of peripheral section 132 can match or be substantially equivalent to the outer diameter or perimeter of proximal end 156 of conduit connector 152. In some instances, inner diameter of the peripheral end 118of VOC 116 can match or be almost the same as the inner diameter of lumen 166 of conduit connector 152 to keep the inner profile as smooth as possible between the two components.

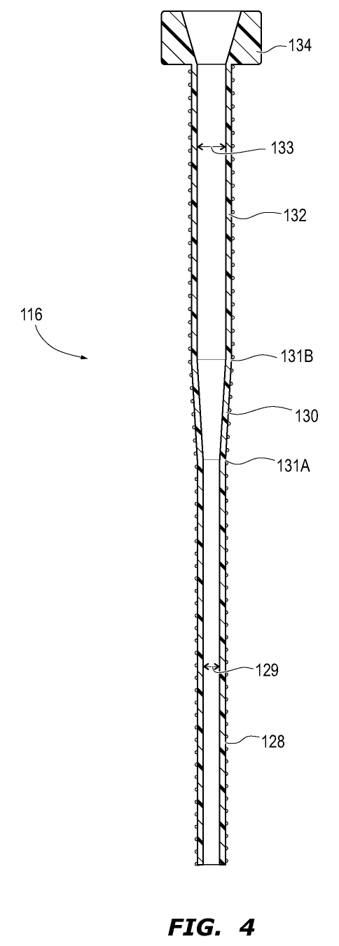

FIG. 4 illustrates a cutaway view of VOC 116 of FIG. 1, according to embodiments described herein. As seen in the illustrated embodiment, VOC 116 can include peripheral section 132, tapering section 130, and central section 128.

As seen in the illustrated embodiment, tapering section 130 can include a taper from a first inner diameter 131A to a second inner diameter 131B.

In some cases, VOC 116 can be or include a braided or reinforcing structure. For instance, in some cases, VOC 116 can include a braided structure or reinforcing braid that extends a length of VOC 116. In some cases, the structure of the reinforcing braid transitions from the first inner diameter to the second inner diameter.

An inner diameter 133 of peripheral section 132 can be 6mm and an inner diameter 129 of central section 128 can be 5 mm. Tapering section 130 can taper from an inner diameter 131A of 6mm to an inner diameter 131B of 5mm. In some cases, tapering section 130 can include a taper angle of between 2 and 3 degrees.

In some cases, the inclusion of a peripheral section 132, tapering section 130, and central section 128 can allow a 20 French sheath to pass over VOC 116 and through a jugular vein of a patient, while still improving internal flow characteristics.

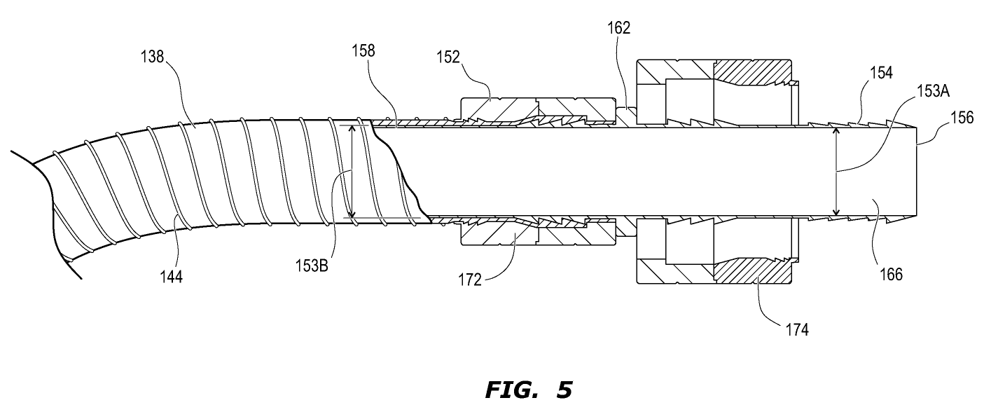

FIG. 5 illustrates a cutaway view of graft component 138 and conduit connector 152 of FIG. 1, according to embodiments described herein. As seen in the illustrated embodiment, graft component 138 can include kink-resistant element 144 and be fluidly connected to conduit connector 152.

As seen in the illustrated embodiment, an inner diameter 153A of the central portion 154 of conduit connector 152 can be similar or the same as an inner diameter 153B of peripheral portion 158.

As previously discussed, kink-resistant element 144 can be formed by a coil and/or sleeve. In some embodiments, the sleeve may extend beyond a distal end of the coil (not shown in FIG. 5). In some instance, the sleeve can be disposed at least partially within the coil and may have an outer diameter substantially the same as the inner diameter of the coil. In other instances, the sleeve may not be disposed within the coil and may only extend from the coil.

Devices, including hub assemblies, catheters, and related components as described above are all within the scope of this disclosure. Additionally, any methods for assembling, forming, and/or bonding the components as described above are likewise within the scope of the present disclosure.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” or “embodiments” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

1. A vascular access system for providing blood flow between two locations in a vascular system of a patient, the vascular access system comprising:
a fluid conduit comprising:
a peripheral portion comprising a first inner diameter; and
a central portion comprising a second inner diameter that is smaller than the first inner diameter.

2. The vascular access system of claim 1, wherein the fluid conduit further comprises a tapering portion disposed between the peripheral portion and the central portion, the tapering portion comprising a taper from the first inner diameter to the second inner diameter.

3. The vascular access system of claim 1, wherein blood is configured to flow through the fluid conduit in a direction of the taper of the tapering portion.

4. The vascular access system of claim 1, wherein the taper comprises a taper angle between 2 and 3 degrees.

5. The vascular access system of claim 1, wherein the tapering portion is at least 5 cm in length.

6. The vascular access system of claim 2, wherein the tapering portion is less than 20 percent of a length of the fluid conduit.

7. The vascular access system of claim 1, wherein a difference between the first inner diameter and the second inner diameter is about 1 mm.

8. The vascular access system of claim 1, wherein the central portion is at least 15 cm in length.

9. The vascular access system of claim 1, wherein the fluid conduit comprises a reinforcing braid that extends a length of the fluid conduit and the reinforcing braid transitions from the first inner diameter to the second inner diameter.

10. The vascular access system of claim 1, further comprising:
a graft conduit configured to be fluidly coupled to the fluid conduit and to the first location in the vascular system;
a connector comprising:
a central portion configured to engage a peripheral end of the fluid conduit;
a peripheral portion configured to engage a central end of the graft conduit;
a lumen extending through the connector, the lumen configured to provide a fluid pathway from the graft conduit to the fluid conduit.

11. The vascular access system of claim 10, wherein the lumen of the connector comprises a constant diameter.

12. A vascular access system for providing blood flow between two locations in a vascular system of a patient, the vascular access system comprising:
a first fluid conduit configured to be fluidly coupled to a first location in the vascular system of the patient, the first fluid conduit comprising a wall structure comprising a self-sealing element;
a second fluid conduit fluidly coupled to the first fluid conduit, the second fluid conduit comprising:
a lumen;
a central end configured to be fluidly coupled to a second location in the vascular system of the patient; and
a tapering portion tapering the lumen from a first diameter to a second diameter; and
a connector comprising:
a peripheral portion configured to engage a central end of the first fluid conduit;
a central portion configured to engage a peripheral end of the second fluid conduit; and
a lumen extending through the connector, the lumen of the connector configured to provide a fluid pathway from the first fluid conduit to the second fluid conduit.

13. The vascular access system of 12, wherein the tapering portion is less than 20 percent of the length of the second fluid conduit.

14. The vascular access system of claim 12, wherein a difference between the first diameter and the second diameter is about 1 mm.

15. The vascular access system of claim 12, wherein second fluid conduit comprises a central portion and a peripheral portion, wherein the tapering portion is disposed between the central portion and the peripheral portion.

16. The vascular access system of claim 11, wherein the lumen of the peripheral portion has the first diameter and the lumen of the central portion has the second diameter.

17. The vascular access system of claim 12, wherein a lumen of the first fluid conduit, the lumen of the connector, and the lumen of the proximal portion each have a diameter equal to the first diameter.

18. The vascular access system of claim 12, wherein the second fluid conduit comprises a reinforcing braid that extends the length of the fluid conduit and transitions from the first diameter to the second diameter.
